(19) United States
(12) Patent Application Publication
(10) Pub. No.: US 2003/0078228 A1
(43) Pub. Date: Apr. 24, 2003

(54) Fas Ligand

(76) Inventors: Doris A. Taylor, Durham, NC (US); Thomas R. Jones, Durham, NC (US)

Correspondence Address: NIXON & VANDERHYE, PC, 1100 N GLEBE ROAD, 8TH FLOOR, ARLINGTON, VA, 22201-4714, US

(21) Appl. No.: 10/252,821

(22) Filed: Sep. 24, 2002

Related U.S. Application Data
(60) Provisional application No. 60/324,041, filed on Sep. 24, 2001.

(51) Int. Cl. A61K 48/00; C12N 5/08

(52) U.S. Cl. 514/44; 424/93.21; 435/366

(57) ABSTRACT

The present invention relates, in general, to Fas Ligand (FasL) and, in particular, to a method of immunoprotecting transplanted cells using an uncleavable form of FasL. The invention also relates to compounds and compositions suitable for use in such a method.

This application claims priority from Provisional Application No. 60/324,041, filed Sep. 24, 2001, the entire content of which is incorporated herein by reference.

### TECHNICAL FIELD

The present invention relates, in general, to Fas Ligand (FasL) and, in particular, to a method of immunoprotecting transplanted cells using an uncleavable form of FasL. The invention also relates to compounds and compositions suitable for use in such a method.

### BACKGROUND

Fas ligand (FasL), a member of the tumor necrosis factor/nerve growth factor family, induces T-cell apoptosis when it binds its receptor, Fas (CD95), on the surface of activated T-cells (Nagata et al, Science 267:1449 (1995)). The presence of FasL in regions of immunoprivilege (such as the eye and testes) suggests that this protein can provide immunoprotection through this interaction. Although FasL is a type II membrane protein, it can be solubilized through cleavage by matrix metalloproteinases (MMPs) (Kayagaki et al, J. Exp. Med. 182:1777 (1995)). This soluble form of FasL (sFasL) not only inhibits the immunoprotective effects of membrane-bound FasL (Tanaka et al, Nature Medicine 4:31 (1998)), but also recruits neutrophils and causes localized inflammation.

The seemingly opposite effects of membrane-bound and soluble FasL have caused a great deal of controversy in the literature regarding the potential of FasL for providing immune protection in allotransplantation in vivo. Both allograft protection and clearance of heterologous cells secondary to massive neutrophil infiltration (Kang et al, Nature Medicine 3:738 (1997)) have been reported. Several hypotheses have been proposed to validate these findings. Takeuchi et al observed that FasL-expressing heart iso- and allografts were rapidly rejected as a result of neutrophil infiltration and edema (Takeuchi et al, The Journal of Immunology 162:518 (1999)). In contrast, FasL expressing liver allografts showed increased survival when transplanted into allogeneic hosts, as compared with untransfected allografts (Li et al, Transplantation 66:1416 (1998)). Furthermore, Lau et al demonstrated immunoprotection of injected islets by cotransplantation with myoblasts transfected with a FasL-expressing plasmid (Lau et al, Science 273:109 (1996)). In contrast, Kang et al observed massive neutrophil infiltration of FasL-expressing islets, resulting in graft destruction (Kang et al, Nature Medicine 3:738 (1997)). Although many hypotheses have been proposed to explain these divergent finding (immunogenecity of viral vectors used for transfection (Kang et al, Nature Medicine 3:738 (1997)) and self apoptosis in transfected cells expressing high levels of FasL (Kang et al, Nature Medicine 3:738 (1997))), it appears neutrophil infiltration of FasL-expressing allografts is a major limiting factor in FasL's ability to provide immune protection. (See Kang et al, Transplantation 69:1813-1817 (2000).) As described herein, in the context of allogeneic myoblast transplantation, high levels of FasL expression in the transplanted cells promote neutrophil recruitment and prohibit allograft survival. However, levels of FasL expression around 5% confer protection on an allograft. The present invention results, in part, from the realization is that in high levels of FasL expression, there is an elevated concentration of sFasL in the region surrounding the allograft. The sFasL recruits neutrophils resulting in the rapid clearance of transplanted cells. However, when the level of expression is reduced to approximately 5%, the cells are protected by the Fas-FasL interaction and the levels of sFasL are sufficiently low that the inflammatory response is minimized. The present invention provides a method of overcoming the unwanted inflammation associated with sFasL by minimizing or preventing cleavage of FasL by MMPs. The present method can be used to immunoprotect a variety of allograft types.

### SUMMARY OF THE INVENTION

The present invention relates generally to FasL and, more specifically, to a method of immunoprotecting transplanted cells using an uncleavable form of FasL. The invention further relates to compounds and compositions suitable for use in such a method.

Objects and advantages of the present invention will be clear from the description that follows.

### BRIEF DESCRIPTION OF THE DRAWINGS

FIGS. 1A and 1B. Immunoblot analysis of FasL, expression by C₂C₁₂ myoblasts. FIG. 1A. Molecular weight marker (Lane 1), FasL positive control (Lane 2), pNeo transfected myoblasts (Lane 3), and pFasL transfected myoblasts (Lane 4). The characteristic 37 kDa FasL band is present only in Lanes 2 and 4. FIG. 1B. Flow cytometry of FasL transfected C₂C₁₂ myoblasts. Analysis showing approximately 15% of the myoblast population, FasL+, expressed FasL. Unstained control myoblasts (shaded area) were not incubated with primary antibody.

FIGS. 2A-2D. Apoptosis of FasL+ myoblasts (TdT staining) as evidenced by FasL-PE staining. FIG. 2A. Activated T-lymphocytes, expressing Fas and FasL, stain positively for apoptosis. FIG. 2B. Undifferentiated FasL+ myoblasts show little or no positive (brown) TdT stain for apoptosis. FIG. 2C. Differentiated FasL+ myoblasts show little apoptosis. FIG. 2D. Differentiated negative control pNeo transfected myoblasts show apoptosis similar to FasL+ myoblasts.

FIGS. 3A and 3B. Fas-producing Jurkat cells incubated with FasL+ or control (pNeo transfected) myoblasts at 3, 6, or 9 hours. FIG. 3A. Percent apoptosis of Fas-positive Jurkat cells at 3, 6, and 9 hours after incubation with FasL+ (ν) or control pNeo (●) myoblasts. FIG. 3B. Percent Jurkat cell viability at 3, 6, and 9 hours after incubation with FasL+ (ν) or control pNeo (●) myoblasts. (Each value represents the mean±SEM of 3 experiments performed in duplicate.)

FIGS. 4A and 4B. Allogeneic mouse Yac-1 T-cells incubated with FasL+ or control pNeo transfected myoblasts for 3, 6, or 9 hours. FIG. 4A. Percent apoptosis of Yac-1 cells 3, 6, or 9 hours after incubation with FasL+ (ν) or control pNeo (●) myoblasts. FIG. 4B. Percent Yac-1 cell viability at 3, 6, or 9 hours after incubation with FasL+ (ν) or control pNeo (●) myoblasts. (Each value represents the mean±SEM of 5 experiments performed in duplicate.)

FIGS. 5A and 5B. Xenogeneic human Molt-A T-cells incubated with FasL+ or control pNeo transfected myoblasts for 3, 6, or 9 hours. FIG. 5A. Percent Molt-A apoptosis 3, 6, or 9 hours after incubation with FasL+ (ν) or control pNeo (●) myoblasts. FIG. 5B. Percent Molt-4 cell viability at 3, 6, or 9 hours after incubation with FasL+ (ν) or control pNeo (●) myoblasts. (Each value represents the mean±SEM of 7 experiments performed in duplicate.)

FIGS. 6A and 6B. Myoblasts engrafted in an allogeneic mouse kidney capsule. FIG. 6A. DAPI-labeled myoblasts (arrows) engrafted in an allogeneic mouse kidney capsule at 3 days after co-injection with 5% FasL+ myoblasts. FIG. 6B. BrdU-labeled FasL+ myoblasts (brown) engrafted in an allogeneic mouse kidney capsule at 3 days after co-injection with 95% FasL-negative myoblasts.

FIGS. 7A-7C. Allogeneic cell survival (number of DAPI-labeled cells per high power field) at 3, 10, and 21 days post injection. Each value represents the mean±SEM for 3 experiments. FIG. 7A. 0% FasL+ myoblast grafts (shaded bars) and 0.05% FasL+ myoblast grafts (white bars). FIG. 7B. 0% FasL+ myoblast grafts (shaded bars) and 5% FasL+ myoblast grafts (white bars). FIG. 7C. 0% FasL+ myoblast grafts (shaded bars) and 25% FasL+ myoblast grafts (white bars). *=p<0.05, a p value of less than 0.05 was considered statistically significant.

FIGS. 8A-8B. Immunoblot analysis of FasL, expression by rabbit skeletal myoblasts. FIG. 8A. Molecular weight marker (Lane 1), FasL positive control (Lane 2), negative control untransfected myoblasts (Lane 3), and pBOSHLFLD4 transfected myoblasts (Lane 4). The characteristic 37 kDa FasL band is present only in Lanes 2 and 4. FIG. 8B. Flow cytometry of FasL transfected rabbit skeletal myoblasts. Analysis showing approximately 15% of the myoblast population, uFasL+, expressed uFasL. Untransfected control myoblasts (shaded area) did not express uFasL.

FIGS. 9A-9C. Apoptosis of uFasL myoblasts (TdT staining) as evidenced by FITC staining. FIG. 9A. Uncleavable FasL+ myoblasts show no positive (green) TdT stain for apoptosis. FIG. 9B. Fixed and permeablised rabbit skeletal myoblasts incubated with DNase I stain positive for apoptosis. FIG. 9C. Untransfected (negative control) myoblasts show no apoptosis.

FIG. 10. Allogeneic cell survival (number of DAPI-labeled cells per high power field) in rabbit myocardium 21 days post injection. Each value represents the mean±SEM. Left. Untransfected allogeneic myoblasts (control). Right. Uncleavable FasL expressing myoblasts. *=p<0.05, a p value of less than 0.05 was considered statistically significant.

FIG. 11. Myoblasts engrafted in an allogeneic rabbit myocardium. uFasL-transfected DAPI-labeled myoblasts engrafted in an allogeneic myocardium. DAPI-labeled myoblasts do not survive when 0% of cells express uFasL.

### DETAILED DESCRIPTION OF THE INVENTION

Cell transplantation, in which a suspension of cells is injected into diseased or injured tissue to induce tissue regeneration, provides a means of treating a wide variety of conditions, including but not limited to diabetes, joint injury, and congenital and acquired muscle diseases (for example, muscular dystrophies, congenital myocardial hypoplasia, myocardial infarction, cardiomyopathy and congestive heart failure). Without immunosuppression, however, only autologous cells are an option for cell transplantation since allogeneic and heterologous cells are rejected via a T-cell mediated response. The present invention provides a method for engineering allogeneic and heterologous cells so that the rejection response can be overcome.

The present method is based on the introduction into cells to be transplanted (including xenogeneic cells) of a construct that, upon expression, results in the production of a form of FasL, or a FasL-like molecule (see, for example, U.S. Pat. No. 6,235,878), that is not subject to cleavage in vivo (e.g., by MMPs) (or at least one subunit thereof is not subject to cleavage) to produce a molecule that inhibits the immunoprotective effects of membrane-bound FasL, or FasL-like molecule, or that recruits neutrophils and causes localized inflammation such that the transplanted cells are destroyed. Such cleavage-resistant molecules are designated herein “uncleavable Fas Ligand” (or “uFasL”).

The “uFasL” of the invention can take the form of naturally occurring FasL (e.g., human FasL (Takahashi et al, Int. Immunol. 6(10):1567 (1994)) modified such that it is not cleavable, for example, by MMP. For instance, in the case of human FasL, the MMP cleavage site can be deleted or mutated (e.g., residues 130-137 be deleted or mutated) (Tanaka et al, Nature Medicine 4(1):31-36 (1998)). Included within the scope of the invention are proteins having an amino acid sequence substantially equivalent (e.g., at least 80%, at least 85%, at least 90%, at least 95%, or at least 99% homologous to human uFasL (using BLAST (or, more specifically, BLOSUM62)) and having a qualitatively equivalent activity (e.g., substantially equivalent in qualitative terms, such as in apoptosis-inducing activity (for example, using the Annexin V-FITC kit described in the Examples). That is, the degree of equivalence can range, for example, from 0.01 to 20 times the activity of human uFasL, preferably 0.2 to 5 times, more preferably 0.5 to 2 times. Non-cleavable forms of the FasL-like molecules of U.S. Pat. No. 6,235,878 can also be used. (Further, see Kang et al, Transplantation 69:1813 (2000).) “uFasL's” suitable for use in the invention can be produced using any of a variety of known recombinant or chemical techniques.

The invention also includes nucleic acid sequences coding for the “uFasL's”, e.g., DNA or RNA sequences (see, for example Tanaka et al, Nature Medicine 4(1):31 (1998)).

The DNA sequences of the invention can be present in a vector, e.g., a plasmid or viral vector (e.g., a retroviral, adenoviral or adeno-associated viral vector). Advantageously, the vector is a non-viral expression vector (for example, pEGSH, pCl-neo, pAdVantage, pMAneo-Luc, pCMS-EGFP, pBOSHFLD-4, pDsRed2-C1, pIRES-hrGFP-1a, gWIZ, phrGFP-N1, pEGFP-N1) in which the “uFasL” encoding sequence is operably linked to a promoter. The promoter used can be any promoter, including a tissue-specific promoter (e.g., a myocyte-specific promoter) so long as it is appropriate for the host cell that is used to effect expression. When the host cell is a human cell, preferred promoters include RSV, CMV, as well as inducible promoters such as reverse tetracycline and ecdysone.

Expression vectors of the invention can further comprise, as necessary, enhancers and/or marker genes. Components of the present vectors are in operable linkage.

Host cells into which the vectors of the invention can be introduced include human and non-human vertebrate cells. When intended for therapeutic use, the host cells can be selected based on the tissue into which the cells are to be transplanted. For example, in the case of diabetes, pancreatic islet cells can be used as host cells, in the case of joint injury, myoblasts or chondrocytes cells can be used, and in the case of myocardial infarction, multiple populations of myoblasts or adult-derived stem cells can be used. However, the host cells are not required to be of the same type as the target tissue. The host cells can be virtually any cell type (e.g., myoblasts transfected with the present vectors can be transplanted for example, with untransfected islet cells, into host tissue (e.g., pancreatic tissue). Either adult-derived stem or progenitor cells, or embryonic stem cells, can be used instead of or in combination with myoblasts or other cells. For example, stem or progenitor cells can be used to give rise to multiple cell types in a given tissue but any or all of the cells can be engineered to express FasL. Further, adipose-derived cells can be used as can marrow-derived cells or adult tissue-derived cells such as muscle cells (progenitor or stem cells).

Introduction of the vector of the invention into the host cells can be effected using techniques well known in the art and using any of a variety of transfection facilitating agents (e.g., liposomal formulations, charged lipids and precipitating agents (e.g., calcium phosphate)) (see Maniatis et al, Molecular Cloning—A Laboratory Manual, Cold Spring Harbor Press (1989)). The vectors can also be introduced as “naked” DNA. Certain applicable techniques are described in the Examples that follow.

Culture of the transformants of the invention can be effected using routinely available techniques and can vary depending on the nature of the host cells.

If appropriate, the “uFasL” can be isolated from the cultured cells and purified using standard protein purification techniques.

Advantageously, the transformed cells of the invention are injected into diseased or injured tissue as a suspension in, for example, normal saline, DMEM, Hyperthermasol, cardioplegia reagents or a solution of human serum albumin.

While optimum conditions can be established by one skilled in the art for any particular patient, generally the number of cells transplanted will be about 3×10⁷ to 1×10⁹. Advantageously, about 5-50% of such cells express a “uFasL” of the invention. The method of delivery can vary depending on the transplantation target but examples include percutaneous delivery, intravascular delivery, intramuscular (skeletal or myocardium) delivery and surgical delivery.

Certain aspects of the invention can be described in greater detail in the non-limiting Examples that follows. In Example 1 (Experimental Details, Verification of FasL overexpression), reference is made to trypsinizing the cells. Harvesting of the cells can also be effected using mechanical disassociation techniques (e.g., scrapping) or using EDTA.

### EXAMPLE I

Experimental Details

Cells

C₂C₁₂ mouse myoblasts were obtained from ATCC. Growth medium consisted of low-glucose DMEM (Gibco), 20% horse serum (Hyclone), and 0.5% v/v Gentamicin (Gibco). Human T-lymphocytes (MOLT-A) (ATCC) were propagated in RPMI 1640 medium supplemented with 10% FBS, 15 mM HEPES buffer, 2 mM L-glutamine, 1.0 mM sodium pyruvate, 0.1 mM non-essential amino acids, 5×10⁻⁵ M β-mercaptoethanol, and penicillin/streptomycin. YAC-1 and mouse T-lymphocytes were obtained from ATCC (160-TIB). Both were cultured in RPML 1640 medium supplemented with 10% FBS and penicillin/streptomycin. Fas-expressing Jurkat cells (Clone E6-l) were obtained from ATCC (TIB-152), and were cultured in 90% RPMI 1640 with 10% FBS, 2 mM L-glutamine, 1.5 g/L sodium bicarbonate, 10 mM HEPES, and 1.0 mM sodium pyruvate.

Overexpression of FasL in C₂C₁₂ Myoblasts

The control, pBCMGS Neo (pNeo), and FasL expressing plasmids, pBCMGS Neo FasL (pFasL), plasmids were obtained from Dr. Adriana Fontana (Lau et al, Science 273:109)) of the University of Zurich, Switzerland. Cells were transfected using a N,N-bis(2-hydroxyethyl)-2-aminoethane sulfonic acid (BES) buffered calcium phosphate precipitation. DNA/calcium phosphate complexes were formed by mixing 150 μl of 0.25 M CaCl₂ with 150 μl of 2×BES-buffered saline (50 mM BES (Sigma), 250 mM NaCl, 1.5 mM Na₂HPO₄, pH 6.95) and 5 μg of either pFasL or pNeo. After 20 minutes, C₂C₁₂ cells plated in 35 mm dishes at 40% confluence were incubated with either pFasL or pNeo at 37° C. and 5% CO₂. After 16 hours, the cells were washed twice with sterile PBS, re-fed with 2 mL growth medium (low glucose DMEM (Gibco), 20% equine serum (Hyclone), 0.5% Gentamicin (Gibco)), and incubated for 24 hours. The cells were then split 1:10 into 35 mm dishes and incubated with 600 μg/mL Geneticin (GIBCO) at 37° C. and 5% CO₂. Media was replenished daily for 4 days, at which time Geneticin was increased to 800 μg/mL for 1 week with daily replenishment. Cells were then allowed to grow without selection for 24 hours, prior to passing and freezing or analysis by immunoblot and flow cytometry for FasL expression. All in vitro and in vivo experiments were performed with newly sorted cells.

Verification of FasL Overexpression

Myoblast transfection efficiency was quantified via Fluorescence Activated Cell Sorting (FACS). Myoblasts were treated with 10 μM KB8301 matrix metalloproteinase inhibitor (Pharmingen) 4 hours prior to harvest to minimize cleavage of sFasL throughout immunodetection of FasL expression. FasL expression was detected in transfected cells with anti-FasL mAB (Pharmingen). Briefly, cells were trypsinized, washed once in wash buffer (PBS containing 1% FBS, 0.1% sodium azide, and KB8301 metalloproteinase inhibitor (BB-2116-00-1, CombiBlocks, Inc., can also be used), resuspended at 2×10⁷ cells/mL, then reacted with anti-FasL antibody (1 μg/50 μl). A biotinylated anti-hamster IgG cocktail secondary antibody (Pharmingen) and a streptavidin conjugated phycoerythrin tertiary antibody (Pharmingen) were bound to the primary antibody (1 μg/100 μL). Cells were then washed twice and analyzed by FACS to obtain those cells expressing FasL. This population of 3×10³ cells was re-tested for FasL expression after 1 week of selection in 800 μg/mL Geneticin.

FasL expression was also confirmed by immunoprecipitation and immunoblot analysis. Monolayers of FasL transfected myoblasts at 80% confluence were incubated with 10 μM matrix metalloproteinase inhibitor KB8301 (Pharmingen) 4 hours prior to harvest to reduce FasL cleavage and sFasL formation. pFasL and pNeo transfected myoblasts plated in 150 mm dishes were washed twice in PBS and lysed with 1 mL RIPA buffer (50 mM Tris (pH 8), 150 mM NaCl (pH 7.4), 1% NP-40, 0.25% sodium deoxycholate, 1 mM EDTA, 5 μL/mL protease inhibitor cocktail (Sigma P8340)). Cell lysate was triturated 30 times and incubated for 1 hour at 4° C.; cellular debris was removed by centrifugation. Lysate supernatant was immunoprecipitated with 5 μL of Antibody Agarose Conjugate (FasL (C-178) AC, Santa Cruz) for 4 hours at 40° C. Each agarose pellet was washed 3 times with RIPA buffer adjusted to 1.0 M NaCl, resuspended in 40 μL SDS sample buffer, and boiled for 5 minutes immediately prior to SDS-polyacrylamide gel electrophoresis. Proteins were electrophoretically transferred to a PVDF membrane (Amersham), incubated with a polyclonal antibody to FasL (F37720, Transduction Laboratories) and developed by ECL plus (Amersham).

Testing of Self Apoptosis in Transfected Myoblasts

C₂C₁₂ cells expressing pFasL or pNeo were grown in 2 wells each on 4 well tissue culture slides (Nunc Sonicseal). One well of each cell type was allowed to grow to confluence, and growth factors were withdrawn to promote myogenic differentiation and myotube formation. The remaining wells were allowed to become 30-40% confluent. Each well was then fixed in 4% formalin, washed in TBS and tested with a commercially available TdT (Terminal deoxynucleotidal Transferase) DNA fragmentation kit (Calbiochem) according to the manufacturer's instruction. Positive and negative controls were provided with the kit and consisted of normal lymphocytes and lymphocytes treated with DNAase.

Co-Culture of Lymphocytes and C₂C₁₂ Myoblasts

Untransfected, pFasL transfected, and pNeo transfected C₂C₁₂ skeletal myoblasts were plated the day before the experiment at a concentration of 3×10⁵ cells/ml. At 16 hours post-plating, when the myoblasts had approximately doubled to 6×10⁵ cells, lymphocytes were centrifuged at 800 rpm for 8 minutes and resuspended at 1×10⁶ cells/mL. The media on the myoblasts was replaced with 1 mL of the lymphocyte solution and incubated at 37° C., 5% CO₂. At 0, 3, 6, and 9 hours, 0.5 ml samples were harvested and tested for early stage apoptosis with a commercially available Annexin V-FITC kit (Oncogene Research Products), and subsequent FACS analysis.

Since necrotic cells, which become porous, can also bind Annexin, counter-staining was done with propidium iodide (PI), which stains both necrotic and late apoptotic cells. By flow cytometry, four populations were defined: viable cells (Annexin negative, PI negative), apoptotic cells (Annexin positive, PI negative), and late apoptotic and necrotic cells (Annexin positive, PI positive). Results are obtained as the percentage of total cells found in each population.

Injection of C₂C₁₂ Cells into the Mouse Kidney Capsule

One day prior to injection, FasL+ myoblasts were labeled with 10 μM bromodeoxyuridine (BrdU) which incorporates into DNA of dividing cells (Ellwart et al, Cytometry 6:513 (1985)). Control C₂C₁₂ cells were fluorescently labeled with 10 μg/mL DAPI for identification. BrdU-positive FasL+ cells were incubated with matrix metalloproteinase inhibitor KB8301 for four hours prior to FACS to sort FasL+ cells as described above. Cells were sorted under sterile conditions to recover a pure population of FasL+ myoblasts. FasL+ myoblasts were mixed at varying percentages into C₂C₁₂ myoblasts at a total concentration of 2×10⁶ cells/50 μL. Injections of DMEM only or 2×10⁶ untransfected cells/50 μL into both allogeneic and syngeneic animals served as control studies.

Animals were sedated with intraperitoneal ketamine (100 mg/kg) and xylazine (15 mg/kg). The right kidney was exposed with a sterile dorsal cut-down under a dissecting microscope. The total volume (50 μL) was injected under the kidney capsule with a Hamilton syringe through a 25 gauge needle. The dorsal incision was repaired and the animals were allowed to recover. Kidneys were excised at 3 days to quantify any early inflammatory response, 10 days to quantify any late inflammatory response, or 21 days to quantify long-term survival.

Tissue Preparation, Histology, and Immunohistochemistry

The left kidney was excised at the specified date (3, 10, or 21 days post-injection). The tissue was placed in PBS and immediately photographed to document the presence, size, and location of any inflammation or abscess. Longitudinally sectioned tissues were fixed in 10% buffered formalin and paraffin embedded for histology. Thin sections were stained with Hematoxylin and Eosin. In addition, sections were stained for BrdU to identify labeled FasL+ myoblasts. Fluorescently labeled (4′,6-diamidino-2-phenylindole dihydrochloride (DAPI) positive) C₂C₁₂ myoblasts were visualized with a fluorescent microscope under a DAPI-filter. The number of surviving DAPI labeled C₂C₁₂ cells was quantified by counting the mean number of labeled cells present per high power field from histologic sections as previously described (Guerette et al, Muscle and Nerve 18:39 (1995)).

Statistical Analysis

All data were compared to control using a student's two tailed t-test. Data were considered statistically significant if p<0.05.

Results

Verification of FasL Expression

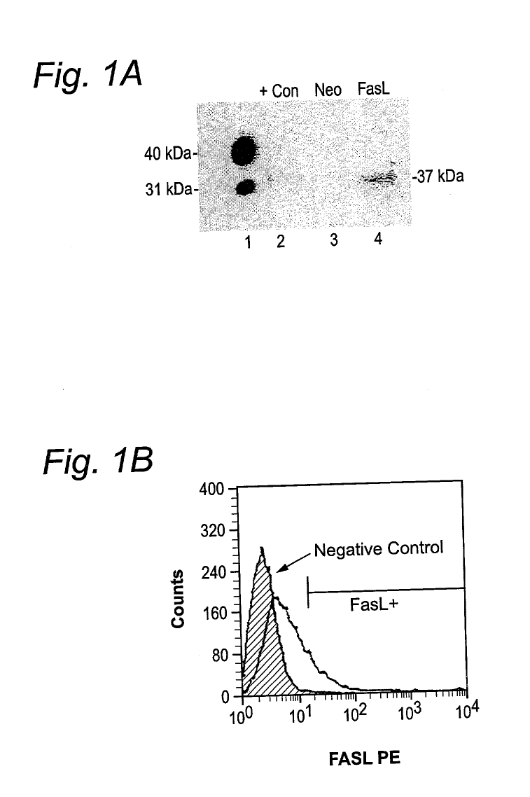

Immunoprecipitation, immunoblotting and FACS were used to show that transfected C₂C₁₂ cells over-express FasL, and to obtain a population limited to FasL+ C₂C₁₂ cells. As depicted in FIG. 1A, immunoprecipitation and subsequent immunoblot analysis of cells transfected with pFasL yielded a 37-kDa FasL protein (Lane 4). In contrast, this band was not seen in C₂C₁₂ cells transfected with pNeo (Lane 3). As indicated by FACS analysis in FIG. 1B, in the presence of KB8301 MMP inhibitor, approximately 15% of the transfected C₂C₁₂ cells expressed FasL (shaded area). In the absence of the inhibitor, only 3-4% of the cell population expressed FasL. C₂C₁₂ cells in the FasL-expressing population were gated and sorted by the flow cytometer to yield a population of 100% FasL expressing cells. These cells were immediately used for in vitro lymphocyte apoptosis, in vivo allotransplantation studies, or frozen for future use.

Detection of Self Apoptosis

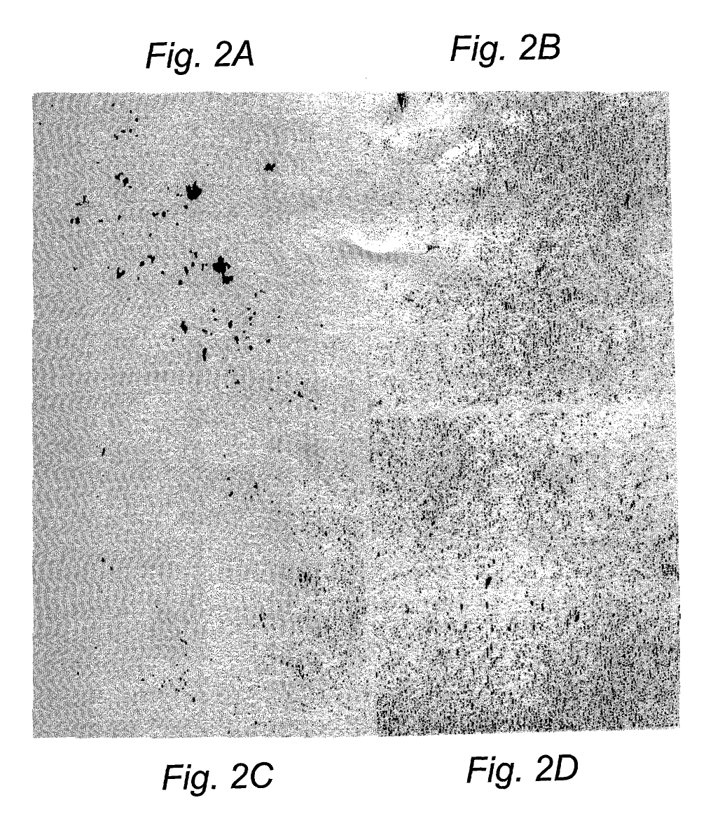

The use of FasL to confer protection of myoblasts in allotransplantation would prove ineffective if myoblasts expressed Fas and underwent Fas/FasL induced apoptosis. Therefore, the extent of self-induced apoptosis was determined in transfected C₂C₁₂ myoblasts by the TdT DNA fragmentation kit (Calbiochem). For comparison, TdT expression in activated lymphocytes is shown in FIG. 2A. As indicated in FIG. 2B, no significant numbers of TdT positive cells (brown) could be found in the undifferentiated myoblasts expressing FasL. It has previously been reported that Fas expression on myoblasts increases with differentiation; thus the extent of self induced apoptosis was also detected in myotubes derived from FasL+ and control pNeo transfected cells. As depicted in FIG. 2C and FIG. 2D, little or no apoptosis was observed in these myotubes.

In vitro FasL Induced T-cell Apoptosis

To determine if FasL+ myoblasts were capable of inducing T-cell apoptosis in vitro, approximately 1×10⁶ activated allogeneic mouse T-cells (Yac-1) or activated xenogeneic human T-cells (Molt-4) were incubated with 1×10⁶ FasL+ or control myoblasts for 3, 6, or 9 hours. The characteristic phosphatidyl serine flip of early stage apoptosis in lymphocytes was measured as described above.

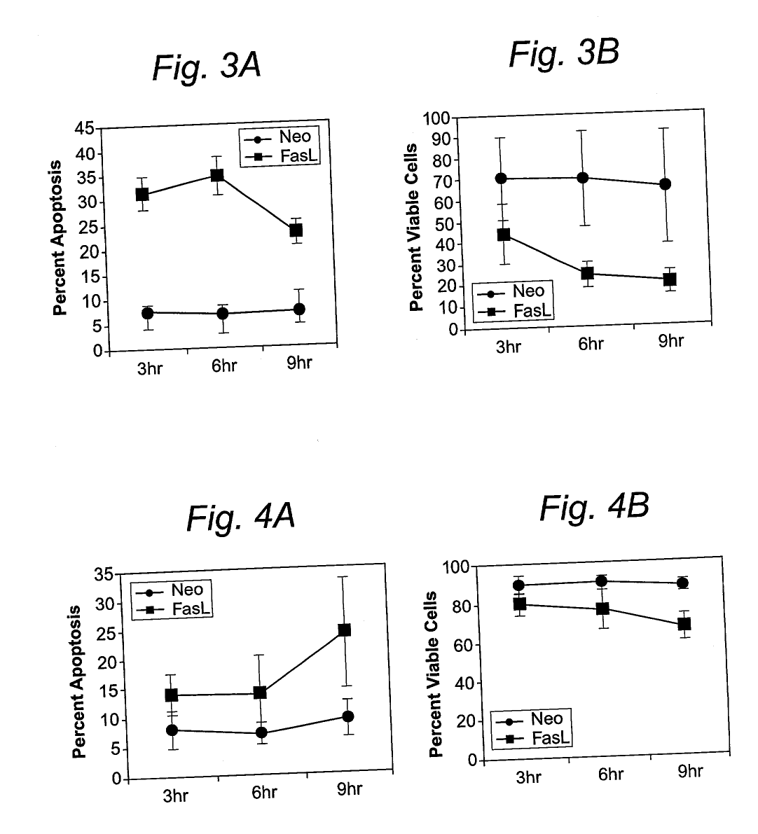

Jurkat cells, which produce Fas, were used as a positive control (FIG. 3). Under the conditions described previously, 31.3+3.38%, 34.7±3.88%, and 23±2.51% of the Jurkat cells underwent apoptosis after 3, 6, and 9 hours, respectively, of incubation with FasL+ myoblasts (FIG. 3A). In comparison, only 7.32±1.52%, 6.65+1.77%, and 7.05±3.93% of Jurkat cells incubated with control pNeo transfected myoblasts underwent apoptosis after 3, 6, and 9 hours, respectively (FIG. 3A). To assess the cumulative apoptotic effect, total cell viability was also measured over the time course of the experiment. Jurkat viability in the presence of pNeo transfected myoblasts remained at near 100% for the entire time course (FIG. 3B). However, when co-cultured with FasL+ myoblasts, Jurkat viability declined from 43.9±14.1%, to 23.9±6.01%, to 19.9±5.25% viability after 3, 6, and 9 hours of incubation, respectively (FIG. 3B).

The ability of FasL to induce apoptosis of activated T-cells has been primarily proposed as a method for allograft protection. Therefore, the ability of FasL+ myoblasts to induce apoptosis in allogeneic Yac-1 T-cells was tested (FIG. 4) at the effector to target ratio where Jurkat cell apoptosis ranged from 20-35%. Under these conditions, early stage apoptosis was elevated at all 3 times in the Yac-1 cells co-cultured with FasL+ myoblasts, above that with the pNeo transfected control cells. At 3, 6, and 9 hours, respectively, Yac-1 apoptosis was measured to be 13.9±3.46%, 13.7+6.63% and 23.9±9.3% (FIG. 4A). The increase in apoptosis of Yac-1 cells incubated with FasL+ C₂C₁₂ myoblasts over 9 hours was also reflected in cell viability data (FIG. 4B), which shows a modest decrease to 79.5±5.92% and 76±10.22%, at 3 and 6 hours, but a more dramatic decrease to 66.6±6.93% by 9 hours. The viability of Yac-1 cells in the presence of control pNeo transfected C₂C₁₂ cells (FIG. 4B) remained relatively constant with a value near 90%.

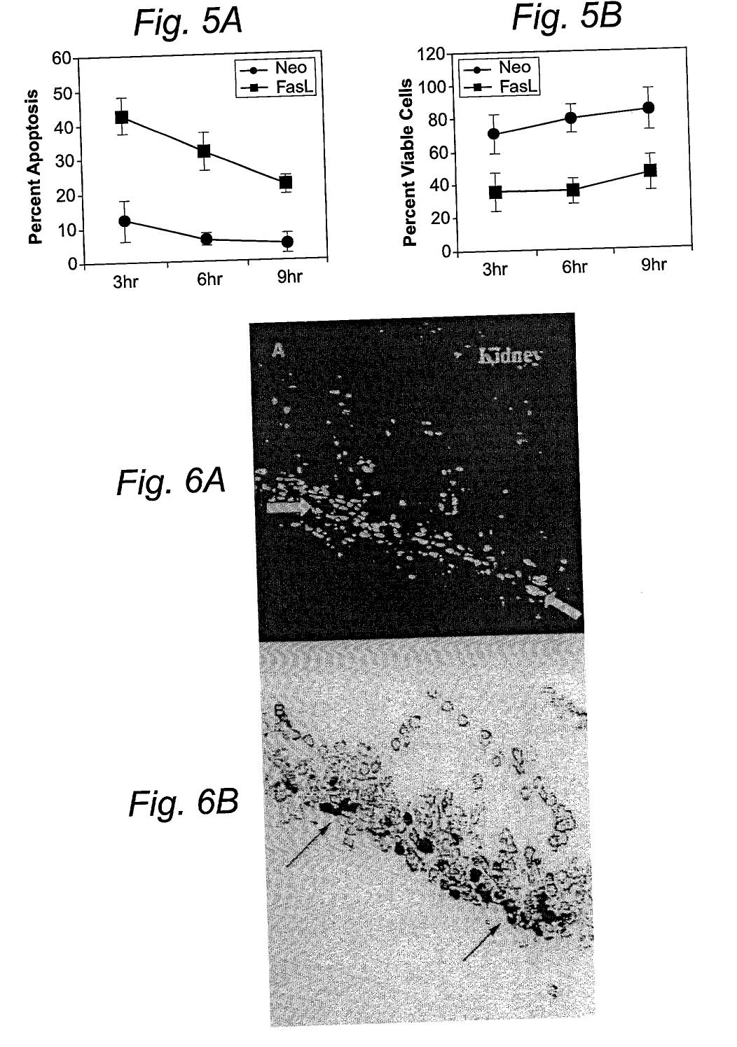

To determine if FasL could have an effect on xenogeneic T-cells, human Molt-4 T-cells were incubated with FasL+ or control pNeo transfected myoblasts (FIG. 5). Surprisingly, apoptosis occurred more rapidly and to a higher degree than with allogeneic cells, or the positive control. Early stage apoptosis was observed at 3, 6, and 9 hour times in 42.7±5.39%, 31.9±5.51%, and 22.0±2.27% of the cells, respectively (FIG. 5A); these percentages are dramatically higher than in control. Furthermore, Fas-FasL induced apoptosis profoundly decreased cell viability as early as 3 hours. As depicted in FIG. 5B, Molt-A cell viability was below 50% by 3 hours. In contrast, Molt-4 cells incubated with control myoblasts, had a cell viability of over 90% for all 9 hours. By 6 and 9 hours of incubation with FasL+ myoblasts, Molt-4 viability was 35.4±7.66% and 46.2±10.8%, respectively.

In vivo Studies

After demonstrating the in vitro apoptotic ability of FasL+ C₂C₁₂, the allogeneic FasL+ myoblasts were transplanted in vivo to see if they could confer allograft protection. However, in vivo there is a relative inability to inhibit MMPs that cleave FasL from the surface of cells to form sFasL. Thus, to attempt to yield various levels of FasL and sFasL in vivo, a titration of FasL+ and FasL-negative C₂C₁₂ cells was performed. To begin to define a level of FasL expression that provides allograft protection in vivo similar to that seen after syngeneic transplant, C₂C₁₂ myoblasts were injected into the kidney capsule of allogeneic and syngeneic mice with varying percentages (0%, 0.05%, 5%, and 25%) of the myoblasts expressing FasL. Untransfected C₂C₁₂ myoblasts were labeled with DAPI, a fluorescent indicator (FIG. 6A). At 3, 10, and 21 days post injection, the number of surviving DAPI-positive cells was quantified by counting the mean number of fluorescent nuclei per high power field. Co-injected BrdU-labeled FasL+ myoblasts were also detected at 3, 10, and 21 days to verify their survival (FIG. 6B).

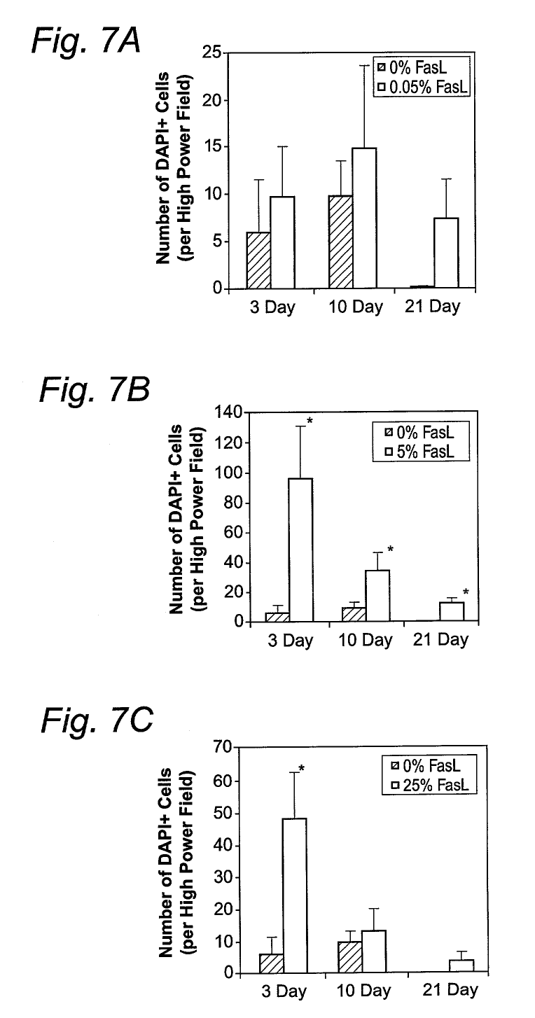

FIG. 7 shows the number of DAPI positive cells present in allogeneic mice at 3, 10, and 21 days post injection. In the control (0% FasL+ cells), the mean number of cells per field was 5.83±5.58, 9.63+3.81 and 0.13±0.09 at 3, 10, and 21 days, respectively. When 0.05% (FIG. 7A) of the injected cells were FasL+, no significant protection of DAPI positive cells was seen compared to control. The mean number of cells per field was 9.67±5.25 (p=0.32), 14.77±8.82 (p=0.31) and 7.3+4.22 (p=0.08), at 3, 10, and 21 days, respectively. By increasing the percentage of FasL+ cells to 5% (FIG. 7B), DAPI positive C₂C₁₂ myoblast survival increased significantly compared to control. At 3 days, the mean was 95.93±35.1 cells (p=0.03), at 10 days, the mean was 34.03±12.35 cells (p=0.05), and at 21 days, the mean was 12.8±2.69 cells (p=0.005). When the percentage of FasL+ cells was increased to 25% (FIG. 7C), at 3 days, the mean number of cells, 47.97±14.44 cells, was significantly higher than control (p=0.03), but was not as high as cell survival with 5% FasL+ myoblasts. Furthermore, when kidneys containing 25% FasL+ cells were excised at 3 days, a large inflammatory region could be seen on the kidney surface. Subsequently, at 10 and 21 days post injection, no protection of DAPI labeled cells was seen. The mean number of surviving cells per field was 13.4±6.76 (p=0.33) and 3.53±2.82 (p=0.15), respectively; when the kidneys were excised, no inflammation was seen.

For comparison, similar populations of FasL+ and C₂C₁₂ myoblasts were injected into syngeneic mice where rejection per se should not occur. The mean cell count per high power field over time was 33.05+6.84, 18.3+5.74 and 12.3±5.73, at 3, 10, and 21 days, respectively. All syngeneic cell counts, regardless of the percentage of FasL+ myoblasts, were statistically equivalent (p>0.05) to this average. When the kidneys from the 25% FasL+ myoblasts injected mice were excised at 3 days, they were inflamed similar to the allogeneic kidneys.

### EXAMPLE II

Experimental Details

Skeletal Muscle Explant

Primary rabbit skeletal myoblasts were isolated from a peripheral soleus muscle biopsy. The biopsy was minced with scissors until a homogeneous mass was obtained. The tissue was plated in a 150 mm polystyrene tissue culture dish (Falcon), fed primary growth media (GM) composed of 10% fetal bovine serum (FBS) (Hyclone), 0.5% Gentamicin reagent (Gibco-BRL) and 89.5% Dulbecco's low glucose Modified Eagle's Medium (DMEM)(Gibco-BRL), and incubated 37° C. humidified atmosphere of 95% air with 5% CO₂.

Skeletal Myoblast Cell Plating and Passaging Technique

Skeletal myoblasts were plated on 150 mm polystyrene tissue culture dishes (Falcon) at a density of 10⁴ cells/cm². GM was replaced every 48 hours. The cells were passaged using a brief exposure to 0.05% trypsin/EDTA when they reached 60% confluence to prevent premature differentiation. Cells were frozen between passages 2 to 4 in primary growth media supplemented with 10% dimethylsulfoxide (DMSO) (Sigma) to cyropreserve. The cells were frozen at a density of 1×10⁶ cells/ml. The cells were stored at −80° C. after reaching this temperature at a rate of 1° C./minute.

Overexpression of Uncleavable FasL in Primary Rabbit Skeletal Myoblasts

On day 0, frozen cells were rapidly thawed and plated in primary GM at a density of 4×10⁴ cells/cm² on 100 mm polystyrene tissue culture dishes. On day 1, cell plating was verified by microscopy and the cells were re-fed with 8 ml of primary GM. On day 2, the cells were transfected with a plasmid expressing the D4 uncleavable FasL mutant, (pBOSHFLD4) (Tanaka et al, Nature Medicine 4(1):31 (1998)). To transfect, the dishes were rinsed twice with Opti-MEM Reduced Serum Medium (Gibco-BRL) and fed with transfection media, which consists of 5% FBS in DMEM. Cells were transfected using the FuGENE (Roche) liposomal transfection reagent according to the manufacturer's protocol. Ten μg of plasmid were used per 100 mm dish, in a FuGENE/plasmid ratio of 3:1. The plasmid-liposome complex was incubated in the cell medium for 24 hours under normal culture conditions of 37° C. humidified atmosphere of 95% air with 5% CO₂. On day 3, the transfection media was removed and the cells were fed with primary GM. Cells were then allowed to grow for 24 hours prior to protein and RNA analysis.

Verification of Uncleavable FasL mRNA

Control and uncleavable FasL expressing primary rabbit skeletal myoblasts were homogenized and total RNA was extracted using a commercially available RNeasy RNA Isolation kit (Qiagen) according to the manufacturer's protocol. RNA concentration was determined by a spectrophotometer. Oligonucleotide primers for the 5′ and 3′ regions of the uncleavable FasL gene were determined using Primer 3 software (MIT). Reverse transcriptase-polymerase chain reaction (RT-PCR) was performed in a MJR Research thermal cycler using the commercially available One-Step RT-PCR kit (Qiagen) following the manufacturer's protocol. PCR products were analyzed on an ethidium bromide-stained 2% agarose gel and photographed under ultraviolet light.

Verification of FasL Overexpression

Myoblast transfection efficiency was quantified via FACS. Uncleavable FasL expression was detected in transfected cells with biotinylated mouse anti-human FasL (Pharmingen). Briefly, cells were trypsinized, washed once in wash buffer (PBS containing 1% FBS and 0.1% sodium azide), resuspended at 2×10⁷ cells/mL, and then reacted with anti-FasL antibody (1 μl/50 μl). A biotinylated anti-mouse IgG secondary antibody (Pharmingen) and a streptavidin conjugated phycoerythrin tertiary antibody (Pharmingen) were bound to the primary antibody (1 μg/100 μl). Cells were then washed and analyzed by FACS to obtain a percentage of cells expressing uncleavable FasL. FasL expression was also confirmed by immunoprecipitation and immunoblot analysis. Monolayers of uncleavable FasL transfected myoblasts at 80% confluence were washed twice in PBS and lysed with RIPA buffer (50 mM Tris [pH 8], 150 mM NaCl [pH 7.4], 1% NP-40, 0.25% sodium deoxycholate, 1 mM EDTA, 5 μl/ml protease inhibitor cocktail [Sigma P8340]). Cell lysate was triturated 30 times with a 21-gauge needle and incubated for 1 hour at 4° C.; cellular debris was removed by centrifugation. Lysate supernatant was immunoprecipitated with 5 μl of Antibody Agarose Conjugate (FasL [C-178] AC, Santa Cruz) for 4 hours at 4° C. Each agarose pellet was washed 3 times with RIPA buffer adjusted to 1.0 M NaCl, resuspended in 40 μl SDS sample buffer, and boiled for 5 minutes immediately prior to SDS-polyacrylamide gel electrophoresis. Proteins were electrophoretically transferred to a PVDF membrane (Amersham), incubated with a polyclonal antibody to FasL (F37720, Transduction Laboratories) and developed by ECL plus (Amersham).

Testing of Self Apoptosis in Transfected Myoblasts

Primary rabbit skeletal myoblasts expressing uncleavable FasL were grown in 2 wells each on 4 well tissue culture slides (Nunc Sonicseal). The remaining wells contained untransfected rabbit skeletal myoblasts. Each well was then fixed in 4% paraformaldehyde and the cells were permeablised in 0.1% sodium citrate, 0.1% Triton-X in PBS. The wells were washed in PBS and tested with a commercially available In situ Cell Death Detection Kit (Roche) according to the manufacturer's instruction.

Injection of Rabbit Skeletal Myoblasts into Allogeneic Myocardium

One day prior to injection, uncleavable FasL (uFasL) transfected and control (untransfected) myoblasts were fluorescently labeled with 10 μg/ml DAPI for identification. To determine the percentage of uFasL expressing cells, a sample of uncleavable FasL transfected myoblasts was analyzed by FACS to obtain a percentage of cells expressing uFasL. Between 10-25% uFasL was deemed appropriate for this experiment.

Cells were harvested under sterile conditions by incubation in 0.05% trypsin/EDTA (Gibco-BRL). The cells were resuspended in DMEM at a concentration of 2×10⁶ cells/200 μl. Populations of untransfected cells served as control studies.

Female New Zealand white rabbits were premedicated-intramuscularly with ketamine (60 mg/kg). The animals were intubated and anesthetized with isoflurane. The heart was exposed through a left thorachotomy in the third intercostal space. The total volume of cells (200 μl) was injected directly into the left ventricular free wall at the apex using a X-gauge needle. The chest was closed and the animals were allowed to recover. After 21 days, the animals were sacrificed by a lethal dose of ketamine, and each heart was harvested and prepared for histologic analysis.

Tissue Preparation, Histology, and Immunohistochemistry

The heart was harvested 21 days post-injection. The tissue was cryoprotected in 30% sucrose/phosphate buffered saline (v/v) and placed at −80° C. Frozen serial sections were cut along the short axis of the heart. DAPI labeled myoblasts were visualized with a fluorescent microscope under a DAPI-filter. The number of surviving DAPI-labeled cells was quantified by counting the mean number of labeled cells present in the region of injection per high power field from histologic sections. Five high power fields were counted from each of 5 frozen sections per heart, resulting in a total of 25 high power fields quantified per heart.

Statistical Analysis

Statistical comparisons between groups were performed by Student's two-tailed test. Differences were considered statistically significant if p<0.05. All data are presented as mean±SEM unless otherwise reported.

Results

Verification of uFasL Overexpression in Primary Rabbit Skeletal Myoblasts

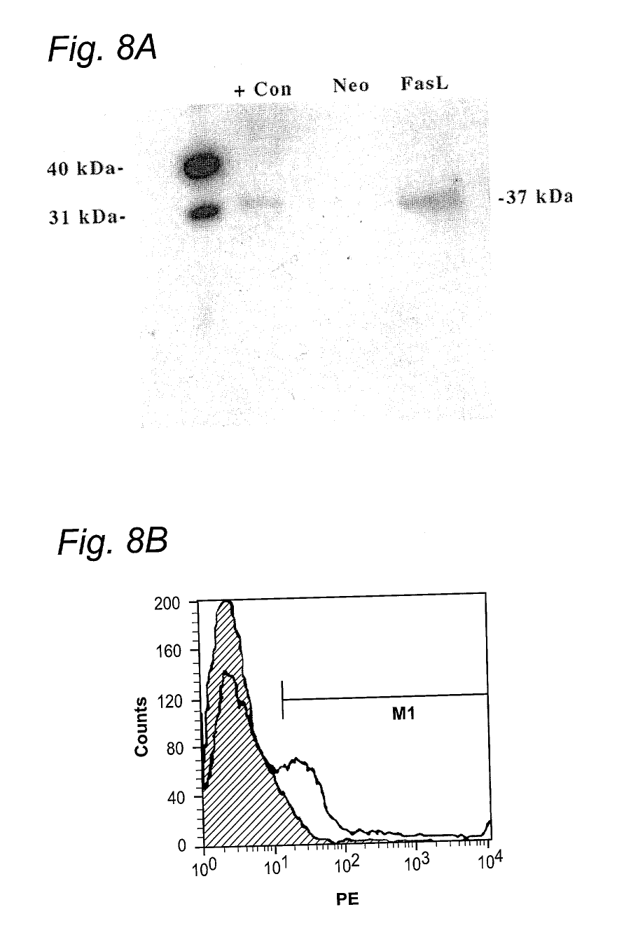

Initially, it was necessary to determine the number of days post-transfection at which the percentage of cells expressing uFasL was maximal. Previous transfection results have shown that this maximum percentage is found two days after transfection. Primary rabbit skeletal myoblasts were transfected and analyzed 2, 3, and 4 days post-transfection. As expected, the maximum percentage of uFasL expressing cells was found two days post-transfection, with a gradual drop in expression over the following days. FIG. 8A shows this drop in percentage for two different populations of myoblasts. This figure also demonstrates that cells from different rabbits often yielded vastly varying transfection efficiencies. FIG. 8B shows the gradual decline in expression in the days post-transfection. Expression on day 2 was 32.29±6.93%, which decreased to 25.35±3.06% on day 3 (p=0.19). Expression dropped further on day 3, to 20.77±2.15% (p=0.12). From this information, it was determined that transfected cells should be used in functional experiments 2 days post-transfection.

Immunoprecipitation, immunoblotting and FACS were used to show that transfected rabbit skeletal myoblasts over-express uFasL. Immunoprecipitation and subsequent immunoblot analysis of cells transfected with pBOSHFLD4 yielded a 37-kDa FasL protein. In contrast, this band was not seen in untransfected rabbit skeletal myoblasts. As indicated by FACS analysis, approximately 15% of the transfected rabbit skeletal myoblasts expressed uFasL in comparison with untransfected cells.

Testing of Self Apoptosis in Transfected Myoblasts

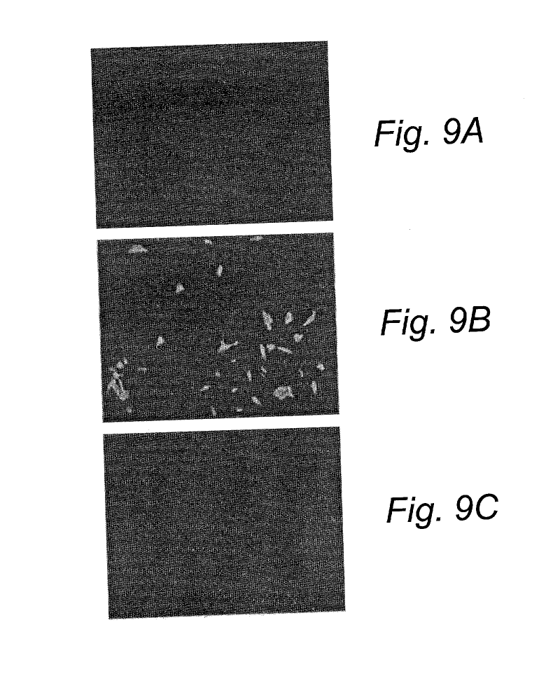

The use of FasL to confer protection of myoblasts in allotransplantation would prove ineffective if myoblasts expressed Fas and underwent Fas/FasL induced apoptosis. Therefore, the extent of self-induced apoptosis was determined in uFasL transfected rabbit skeletal myoblasts by TUNEL staining (for DNA strand breaks) using the In Situ Cell Death Detection Kit (Roche). As indicated in FIG. 9, no positive cells (green) could be found in the undifferentiated rabbit skeletal myoblasts expressing FasL (FIG. 9A). For comparison, TUNEL expression in fixed, permeablised myoblasts treated with DNase (positive control) is shown in FIG. 9B.

In vivo Studies

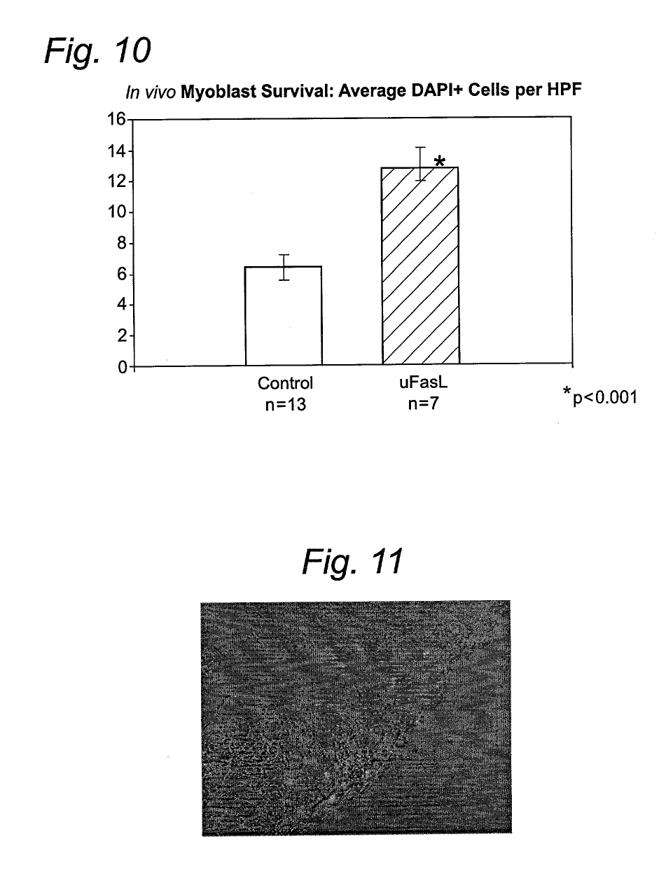

After verifying uFasL overexpression on myoblasts and proving these myoblasts do not undergo self-apoptosis, allogeneic uFasL-expressing myoblasts were transplanted in vivo to determine their ability to confer allograft protection. Rabbit skeletal myoblasts were injected into allogeneic myocardium near the apex of the heart. All injected myoblasts were labeled with DAPI, a fluorescent indicator. At 21 days post injection, hearts were harvested and cryosectioned, and the number of surviving DAPI-positive cells was quantified by counting the mean number of fluorescent nuclei per high power field (see FIG. 10).

FIG. 11 shows the number of DAPI positive cells present in allogeneic myocardium 21 days post injection. In the control animals (injected with untransfected myoblasts), the mean number of cells per field was 5.61±0.81 at 21 days. In the experimental animals (injected with uFasL transfected myoblasts), the mean number of cells per field was 12.79±1.31 at 21 days.

### EXAMPLE III

A method for delivery of plasmid vector to cells is described as follows.

Rabbit skeletal myoblast cells were obtained from a biopsy of a rabbit soleus muscle. Cells were proliferated by tissue culture techniques and frozen in a solution of 89.5% FBS, 10% DMSO, and 0.5% Gentamicin at −80° C. Cells were rapidly thawed and in a 37° C. water bath and plated on 35 mm tissue culture dishes in 1 mL 5% FBS DMEM media. Immediately after plating the lipid/DNA mixture (consisting of 2 μg plasmid and 10 μg GeneLIMO-Plus cationic lipid) was introduced. Three hours after plating, 1 mL growth medium (10% FBS in DMEM with 0.5% Gentamicin) was introduced to the plate. Twenty four hours post-transfection, the cells were fed with growth medium. Transfection levels peaked after 48 hours at 30%. This indicates a three-fold increase over previous transfection efficiency levels.

All documents cited above are hereby incorporated in their entirety by reference.

What is claimed is:

1. A method of rendering mammalian cells resistant to rejection upon transplantation into a mammalian host comprising introducing into said cells a construct comprising a nucleic acid encoding a form of a Fas Ligand (FasL) or FasL-like molecule that is not subject to cleavage by an enzyme of said host to yield a molecule: (i) that inhibits an immunoprotective effect of membrane-bound FasL, or FasL-like molecule, or (ii) that recruits neutrophils of said host, wherein said introduction is effected under conditions such that said nucleic acid is expressed and said FasL or FasL-like molecule is thereby produced.

2. The method according to claim 1 wherein said host is a human.

3. The method according to claim 2 wherein said FasL is human FasL the matrix metalloprotein (MMP) cleavage site of which is deleted or mutated.

4. The method according to claim 1 wherein the cells are human cells.

5. The method according to claim 1 wherein said cells are pancreatic islet cells, myoblasts, chondrocytes or stem cells.

6. The method according to claim 1 wherein said nucleic acid is present in a vector.

7. The method according to claim 6 wherein said vector is a plasmid or viral vector.

8. The method according to claim 1 wherein said nucleic acid is operately linked to a promoter.

9. The method according to claim 8 wherein said promoter is a tissue specific promoter.

10. The method according to claim 8 wherein said promoter is an inducible promoter.

11. A cell comprising a construct comprising a nucleic acid encoding a form of a Fas Ligand (FasL) or FasL-like molecule that is not subject to enzymatic cleavage to yield a molecule: (i) that inhibits an immunoprotective effect of membrane-bound FasL, or FasL-like molecule, or (ii) that recruits neutrophils.

12. A method of immunoprotecting a population of cells transplanted into a patient comprising introducing into a number of cells of said population, prior to transplantation, a construct comprising a nucleic acid encoding a form of a Fas Ligand (FasL) or FasL-like molecule that is not subject to cleavage by an enzyme of said patient to yield a molecule: (i) that inhibits an immunoprotective effect of membrane-bound FasL, or FasL-like molecule, or (ii) that recruits neutrophils,
wherein said introduction is effected under conditions such that said nucleic acid is expressed, and
wherein said number of cells is sufficient such that said population of cells is immunoprotected.

13. The method according to claim 12 wherein said population of cells, or subpopulation thereof, comprises allogeneic or heterologous cells.

14. The method according to claim 12 wherein said population of cells, or subpopulation thereof, comprises pancreatic islet cells, myoblasts, chondrocytes or stem cells.

15. The method according to claim 12 wherein said number of cells represents at least 5% of said population.
